(19) United States
(12) Patent Application Publication
(10) Pub. No.: US 2013/0324563 A1
(43) Pub. Date: Dec. 5, 2013

(54) METHOD OF INTENSIFYING THE PHYSIOLOGICAL ACTION OF CAFFEINE

(71) Applicants: Takeshi Kokubo, Yokohama-shi, (JP); Masaya Kanayama, Yokohama-shi (JP); Kyoko Tazumi, Yokohama-shi (JP); Yuri Tamaki, Yokohama-shi (JP)

(72) Inventors: Takeshi Kokubo, Yokohama-shi, (JP); Masaya Kanayama, Yokohama-shi (JP); Kyoko Tazumi, Yokohama-shi (JP); Yuri Tamaki, Yokohama-shi (JP)

(73) Assignee: Kirin Holdings Kabushiki Kaisha

(21) Appl. No.: 13/995,581

(22) PCT Filed: Dec. 14, 2011
(86) PCT No.: PCT/JP2011/006978

§ 371 Date: Jul. 30, 2013

(30) Foreign Application Priority Data
Dec. 21, 2010 (JP) 2010-284678

(51) Int. Cl. A61K 31/522; A61K 31/198

(52) U.S. Cl. CPC A61K 31/522; A61K 31/198
USPC 514/263.31

(57) ABSTRACT

It is an object of the present invention to provide: a method for safely and effectively enhancing the physiological action of caffeine even from a low intake of caffeine, when utilized various types of a physiological action of a caffeine which involve stimulation of the central nerve to promote mental function; and a composition for improving physiological function, which comprises, as an active ingredient, the caffeine whose physiological action has been enhanced. As a means to solve the above-mentioned object, the physiological action of caffeine is enhanced by combining caffeine with ornithine or a salt thereof. In addition, the ornithine or a salt thereof in an amount equivalent to 0.1 to 100 parts by weight of free ornithine based on 1 part by weight of the caffeine is mixed with caffeine, as an agent for enhancing the physiological action of caffeine, thereby providing a composition for improving a physiological function comprising caffeine as an active ingredient. The improvement of a physiological function by caffeine in the present invention is the improvement of a physiological function by mental fatigue-reducing action, drowsiness-preventing action, or concentration-maintaining action.

### TECHNICAL FIELD

The present invention relates to a method for safely and effectively enhancing various types of physiological action of caffeine which involve stimulation of the central nerve to promote mental function; and a composition for improving a physiological function, which comprises, as an active ingredient, the caffeine whose physiological action has been enhanced.

### BACKGROUND ART

Caffeine is one type of purine alkaloid, and is abundantly contained in coffee, green tea, red tea, oolong tea, cola, chocolate and the like. Generally, caffeine has been known to have physiological action to stimulate the central nerve to promote mental function. Thus, many people consume caffeine-containing food and drink products for purposes such as the amelioration of mental fatigue, prevention of drowsiness, and the maintenance of concentration during working, driving, and so on.

However, if people consume caffeine for the above-mentioned purposes, it is necessary for consume large quantities of caffeine-containing food and drink products. Moreover, since caffeine has a short half-life in a body, it is necessary to intake caffeine frequently in order to maintain the desired effects. Caffeine has been problematic in that if it is frequently consumed in a large amount, it causes side effects such as insomnia and dizziness. Furthermore, caffeine has been also problematic in that when a person who customarily takes caffeine does not take it for 0.5 to 1 day, symptoms such as headache, anxiety, feeling of fatigue, and the lack of concentration would appear. Accordingly, has been desired to establish a method for obtaining the same effects from caffeine, even if the intake of caffeine is reduced, and a method for sustaining the effects of caffeine.

In order to improve the physiological function of caffeine, various attempts have been conventionally made to mix caffeine with substances other than caffeine. For instance, Japanese unexamined Patent Application Publication No. 2002-281940 discloses an agent for improving energy metabolism during exercise, in which caffeine is combined with fructose to achieve reduction in the consumption of muscle glycogen and promotion of the burning of body fat during exercise such as sports. In addition, Japanese unexamined Patent Application Publication No. 2002-322063 discloses a mental fatigue-reducing composition, a concentration maintaining and enhancing composition, and a mental vitality maintaining and enhancing composition, each of which comprises, as an active ingredient, a mixed component consisting of caffeine, theanine and arginine, and is used to reliably and effectively achieve the improvement of mental function.

Moreover, Japanese unexamined Patent Application Publication No. 2003-33156 discloses a stimulating food product maintaining stimulating action for a long period of time, wherein the stimulating food product is produced by mixing coffee extract, rosemary extract, honey and caffeine with sugar to process the mixture into a solid form. Japanese unexamined Patent Application Publication No. 2007-119349 discloses a hepatic lipid accumulation-suppressing agent and a serum lipid-suppressing agent, which each comprise caffeine and epigallocatechin gallate, and have an enhanced hepatic lipid accumulation-suppressing effect and an enhanced serum lipid suppressing effect, respectively. Japanese unexamined Patent Application Publication No. 2007-153816 discloses a fatigue-improving composition comprising 2-aminoethanesulfonic acid, vitamin B, caffeine and capsaicin, and having an enhanced fatigue-improving action.

Furthermore, Japanese unexamined Patent Application Publication No. 2008-63281 discloses a composition for oral administration, which comprises caffeine and Osei (Polygonatum rhizome) extract derived from Polygonatum, a Liliaceae plant, and is used to induce the consciousness level of the brain to a relaxed condition. Japanese unexamined Patent Application Publication No. 2009-106253 discloses functional coffee, which comprises caffeine and taurine and has an improved effect of enhancing endurance during exercise and recovering from fatigue. Japanese unexamined Patent Application Publication No. 2010-195761 discloses a drowsiness-preventing composition, which comprises caffeine, pepper extract, ginger extract and arginine, and has an enhanced effect of preventing drowsiness.

As described above, the mixing of substances other than caffeine has been studied in order to improve various types of physiological action of caffeine. However, such mixing of substances other than caffeine has been problematic in that it could not sufficiently improve the physiological action of caffeine or in that substances to be mixed have too strong stimulation. Accordingly, under the current circumstances, satisfactory results could not be necessarily obtained.

### PRIOR ART DOCUMENTS

### Patent Documents

Patent Document 1: Japanese unexamined Patent Application Publication No. 2002-281940
Patent Document 2: Japanese unexamined Patent Application Publication No. 2002-322063
Patent Document 3: Japanese unexamined Patent Application Publication No. 2003-33156
Patent Document 4: Japanese unexamined Patent Application Publication No. 2007-119349
Patent Document 5: Japanese unexamined Patent Application Publication No. 2007-153816
Patent Document 6: Japanese unexamined Patent Application Publication No. 2008-63281
Patent Document 7: Japanese unexamined Patent Application Publication No. 2009-106253
Patent Document 8: Japanese unexamined Patent Application Publication No. 2010-195761

### SUMMARY OF THE INVENTION

### Object to be Solved by the Invention

It is an object of the present invention to provide: a method for safely and effectively enhancing the physiological action of caffeine, in which effective action effects can be obtained even from a low intake of caffeine and the sustained effects can also be obtained from caffeine, when utilized various types of physiological action of the caffeine which involve stimulation of the central nerve to promote mental function; and a composition for improving a physiological function comprising, as an active ingredient, the caffeine whose physiological action has been enhanced.

### Means to Solve the Object

The present inventors have conducted intensive studies regarding a method for safely and effectively enhancing the physiological action of caffeine used as an active ingredient, when utilized various types of physiological action of the caffeine. As a result, the inventors have found that effective action effects can be obtained even from a low intake of caffeine by combining the caffeine with ornithine, and also that the sustained effects of caffeine can be obtained. Thus, the inventors have found that it is possible to safely and effectively enhance the physiological action of caffeine by the coexistence of such caffeine with ornithine, thereby completing the present invention.

Specifically, the present invention consists of: a method for enhancing physiological action of caffeine, comprising combining caffeine with ornithine or a salt thereof; or a composition for improving a physiological function comprising caffeine as an active ingredient, comprising ornithine or a salt thereof in an amount corresponding to 0.1 to 100 parts by weight of free ornithine based on 1 part by weight of caffeine, mixed with caffeine, as an agent for enhancing physiological action of caffeine.

The mixing ratio of caffeine and ornithine or a salt thereof in the present invention is set for the ornithine or a salt thereof to be in an amount corresponding to 0.1 to 100 parts by weight of, preferably 0.5 to 50 parts by weight of, and more preferably 1 to 10 parts by weight of free ornithine based on 1 part by weight of caffeine. In the present invention, the physiological action of caffeine may be mental fatigue-reducing action, drowsiness-preventing action or concentration-maintaining action.

The present invention includes a composition for improving physiological function comprising caffeine as an active ingredient, wherein the improvement of physiological function by caffeine is the improvement of physiological function by the mental fatigue-reducing action, drowsiness-preventing action, or concentration-maintaining action.

That is, the present invention specifically consists of: (1) a method for enhancing the physiological action of caffeine, comprising combining caffeine with ornithine or a salt thereof; (2) the method for enhancing physiological action of caffeine according to (1) above, wherein caffeine is combined with ornithine or a salt thereof in an amount equivalent to 0.1 to 100 parts by weight of free ornithine based on 1 part by weight of caffeine; (3) the method for enhancing physiological action of caffeine according to (1) or (2) above, wherein the physiological action of caffeine is mental fatigue-reducing action, drowsiness-preventing action, or concentration-maintaining action; (4) a composition for improving a physiological function comprising caffeine as an active ingredient, wherein ornithine or a salt thereof is added to caffeine as an agent for enhancing physiological action of caffeine in an amount corresponding to 0.1 to 100 parts by weight of free ornithine based on 1 part by weight of caffeine; and (5) the composition for improving a physiological function comprising caffeine as an active ingredient according to (4) above, wherein the improvement of a physiological function by caffeine is the improvement of a physiological function by mental fatigue-reducing action, drowsiness-preventing action, or concentration-maintaining action.

### Effect of the Invention

According to the present invention, when utilized various types of physiological action of caffeine which involve stimulation of the central nerve to promote mental function, such as mental fatigue-reducing action, drowsiness-preventing action or concentration-maintaining action, effective action effects can be obtained even from a low intake of caffeine, and further, the sustained action effects of caffeine can be obtained. Accordingly, the present invention provides: a method for safely and effectively enhancing the physiological action of caffeine; and a composition for improving a physiological function comprising, as an active ingredient, the caffeine whose physiological action has been enhanced.

### BRIEF DESCRIPTION OF DRAWINGS

FIG. 1 is a view showing a summary of an intake test carried out in Examples of the present invention.

FIG. 2 shows the results of a change in POMS “vigor” in an intake test carried out in Examples of the present invention. In the figure, “Pla” indicates a placebo intake group, and “CFN” indicates a caffeine intake group. In addition, “CFN+ORH” indicates a group in which both caffeine and ornithine hydrochloride were taken in combination. The horizontal axis indicates “time”, and the longitudinal axis indicates “vigor”.

FIG. 3 shows the results of a change in POMS “fatigue” in an intake test carried out in Examples of the present invention. In the figure, definitions regarding groups are the same as those in FIG. 2. The horizontal axis indicates “time”, and the longitudinal axis indicates “fatigue level”.

FIG. 4 shows the results of a change in POMS “confusion” in an intake test carried out in Examples of the present invention. In the figure, definitions regarding groups are the same as those in FIG. 2. The horizontal axis indicates “time”, and the longitudinal axis indicates “confusion level”.

FIG. 5 shows the results of a change in VAS “drowsiness” in an intake test carried out in Examples of the present invention. In the figure, definitions regarding groups are the same as those in FIG. 2. The horizontal axis indicates “time”, and the longitudinal axis indicates “drowsiness”.

FIG. 6 shows the results of a change in VAS “feeling of fatigue” in an intake test carried out in Examples of the present invention. In the figure, definitions regarding groups are the same as those in FIG. 2. The horizontal axis indicates “time”, and the longitudinal axis indicates “feeling of fatigue”.

FIG. 7 shows the results of a change in VAS “concentration” in an intake test carried out in Examples of the present invention. In the figure, definitions regarding groups are the same as those in FIG. 2. The horizontal axis indicates “time”, and the longitudinal axis indicates “concentration level”.

FIG. 8 shows the results of a change in VAS “vigor” in an intake test carried out in Examples of the present invention. In the figure, definitions regarding groups are the same as those in FIG. 2. The horizontal axis indicates “time”, and the longitudinal axis indicates “vigor”.

FIG. 9 shows the results of a change in VAS “willingness to working” in an intake test carried out in Examples of the present invention. In the figure, definitions regarding groups are the same as those in FIG. 2. The horizontal axis indicates “time”, and the longitudinal axis indicates “willingness level to working”.

FIG. 10 shows the results of a change in SIgA contained in saliva in an intake test carried out in Examples of the present invention. In the figure, definitions regarding groups are the same as those in FIG. 2. The horizontal axis indicates “time”, and the longitudinal axis indicates “the amount of SIgA in saliva”.

### MODE OF CARRYING OUT THE INVENTION

The present invention consists of: a method for enhancing the physiological action of caffeine, comprising combining caffeine with ornithine or a salt thereof; or a composition for improving a physiological function comprising caffeine as an active ingredient, comprising ornithine or a salt thereof in an amount equivalent to 0.1 to 100 parts by weight of free ornithine based on 1 part by weight of caffeine, mixed with caffeine, as an agent for enhancing physiological action of caffeine.

The caffeine used in the present invention may be either a hydrate or an anhydride. The ornithine contacted with caffeine in the present invention may be either L-ornithine or D-ornithine. Of these, L-ornithine is preferably used. Such ornithine may be obtained by a method such as a chemical synthesis method, a fermentative production method, or a method for extracting ornithine from a certain material, and may be then used. Alternatively, a commercially available ornithine may also be used.

Such ornithine may be chemically prepared by the method described, for example, in Coll. Czechoslov. Chem. Commun., 24, 1993 (1959), or may also be prepared by the fermentative production described in Japanese unexamined Patent Application Publication No. 53-24096, Japanese unexamined Patent Application Publication No. 61-119194, etc. Otherwise, a commercially available ornithine product may also be used.

Examples of ornithine salts include acid addition salts, metal salts, ammonium salts, organic amine addition salts, and amino acid addition salt.

Examples of the acid addition salts include: inorganic acid salts such as hydrochloride, sulfate, nitrate, or phosphate; and organic acid salts such as acetate, maleate, fumarate, citrate, malate, lactate, α-ketoglutarate, gluconate, or caprylate. Examples of the metal salts include: alkali metal salts such as sodium salt or potassium salt; alkaline earth metal salts such as magnesium salt or calcium salt; aluminum salts; and zinc salts. Examples of the ammonium salts include the salts of ammonium and tetramethylammonium. Examples of the organic amine addition salts include the salts of morpholine and piperidine. Examples of the amino acid addition salts include the salts of glycine, phenylalanine, lysine, aspartic acid, and glutamic acid. Among the above described ornithine salts, hydrochloride, citrate, malate, α-ketoglutarate, aspartate are preferably used. Such salts may be used appropriately in combination with other salts. Moreover, two or more types of salts may be appropriately combined and then used.

The physiological action of caffeine can be enhanced by combining caffeine with ornithine or a salt thereof. The thus enhanced physiological action of caffeine includes a mental fatigue-reducing effect, a drowsiness-preventing effect, and a concentration-maintaining effect. An example of a method comprising combining caffeine with ornithine or a salt thereof is a method comprising adding ornithine to caffeine and then mixing them.

The amount of ornithine or a salt thereof combined with caffeine is an amount equivalent to generally 0.1 to 100 parts by weight, preferably 0.5 to 50 parts by weight, and more preferably 1 to 10 parts by weight of free ornithine based on 1 part by weight of caffeine.

In order to safely and effectively enhance the physiological action of caffeine, as necessary, additives suitable for each intended use may also be appropriately combined with caffeine and ornithine or a salt thereof. Examples of such additives include: amino acids such as theanine, valine, leucine, isoleucine, arginine, lysine, glutamine, serine, glycine, cysteine, or threonine; cool feeling stimulators such as menthol; and hot feeling stimulators such as pepper.

Moreover, as necessary, the following substances may also be combined with caffeine and ornithine or a salt thereof: a wheat-derived component, malt, wort, hop, hop extract, fresh tea leaf extract, brown rice, oats, senna tea, corn, collagen peptide, deep ocean water for drinking, dry rose hip, black bean, black sesame, Aloe arborescens, peel of Citrus junos, vitamin such as vitamin C, spice extract, red tea, fructose-glucose liquid sugar, sugar, common salt, citrulline, organic acid such as citric acid, calcium lactate, iron pyrophosphate, calcium gluconate, potassium chloride, magnesium chloride, acidulant, skimmed milk powder, milk protein, milk peptide, cyclodextrin, etc.

The physiological action of caffeine can be enhanced by combining caffeine with ornithine or a salt thereof. Thereby, the content of caffeine in a composition directed to achieving the physiological action can be reduced.

For example, by adding ornithine or a salt thereof to a preparation containing caffeine as an active ingredient (hereinafter also referred to as a caffeine preparation), there can be produced a caffeine preparation whose necessary dose has been reduced, in comparison with a case in which such ornithine or a salt thereof is not added. Such a caffeine preparation can be produced by mixing caffeine, ornithine or a salt thereof, and as necessary, a carrier and the like, according to any given method that is well known in the technical field of pharmaceutics.

For example, a caffeine preparation is produced using additives such as an excipient, a binder, a disintegrator, a lubricant, a dispersant, a suspending agent, an emulsifier, a diluent, a buffer, an anti-oxidant and a microbial inhibitor, as well as caffeine and ornithine or a salt thereof.

The present invention will be more specifically described in the following examples. However, these examples are not intended to limit the scope of the present invention.

### Example 1

(1) Crystalline cellulose alone, or 100 mg of caffeine or 250 mg of ornithine hydrochloride (which contained 200 mg of ornithine) with crystalline cellulose, was filled into each capsule, so as to prepare hard capsule No. 1. The weight of each capsule was adjusted to be uniform by adding crystalline cellulose. As a placebo food product, two capsules containing crystalline cellulose alone were used. As a caffeine food product, one capsule containing 100 mg of caffeine and one capsule containing crystalline cellulose alone were used. As a food product containing both caffeine and ornithine hydrochloride, one capsule containing 100 mg of caffeine and one capsule containing 250 mg of ornithine hydrochloride were used.
(2) Nineteen healthy male or female persons of 27 to 45 years old were tested as subjects. Each subject was placed at rest for 30 minutes or more in the morning, and saliva was then recovered from each subject at 9:30 a.m. Thereafter, each subject responded to POMS (Profile of Mood State) and VAS (Visual Analog Scale) questionnaires. Subsequently, each subject ingested a test food. Thirty minutes after the ingestion of a test food, each subject underwent a simple calculation load test for 30 minutes. This test was defined as a mental workload.

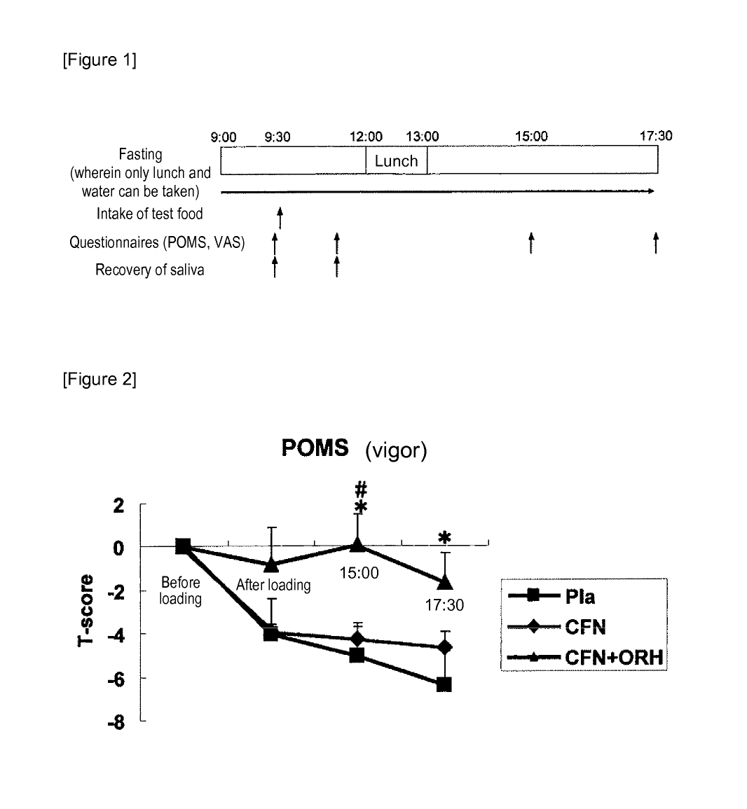

When the subjects took capsules as test foods, the capsules were prepared, such that the types of test foods could not be determined based on the taste and shape of each capsule. The test was carried out as a crossover double blind test performed on three groups. After completion of the calculation load, saliva was recovered from each subject, and the subjects then responded to the POMS and VAS questionnaires. Thereafter, the subjects responded to the POMS and VAS questionnaires at 15:00 p.m. and 17:30 p.m. Based on the VAS questionnaires, “drowsiness”, “feeling of fatigue”, “concentration”, “vigor”, and “willingness to working” were evaluated. A summary of the test is shown in FIG. 1. This test was carried out once for each of the three types of test foods. On the test day, the subjects were refrained from ingestion of any foods and beverages, except for the designated lunch and water.

The results of the test are shown in FIGS. 2 to 9. The paired t-test was applied for statistical processing. The item that showed a significant difference of p<0.05 in comparison with the placebo intake group was indicated with the symbol *, and the item that showed a significant difference of p<0.01 in comparison with the placebo intake group was indicated with the symbol **. In addition, the item that showed a significant difference of p<0.05 in comparison with the caffeine intake group was indicated with the symbol #, and the item that showed a significant difference of p<0.01 in comparison with the caffeine intake group was indicated with the symbol ##.

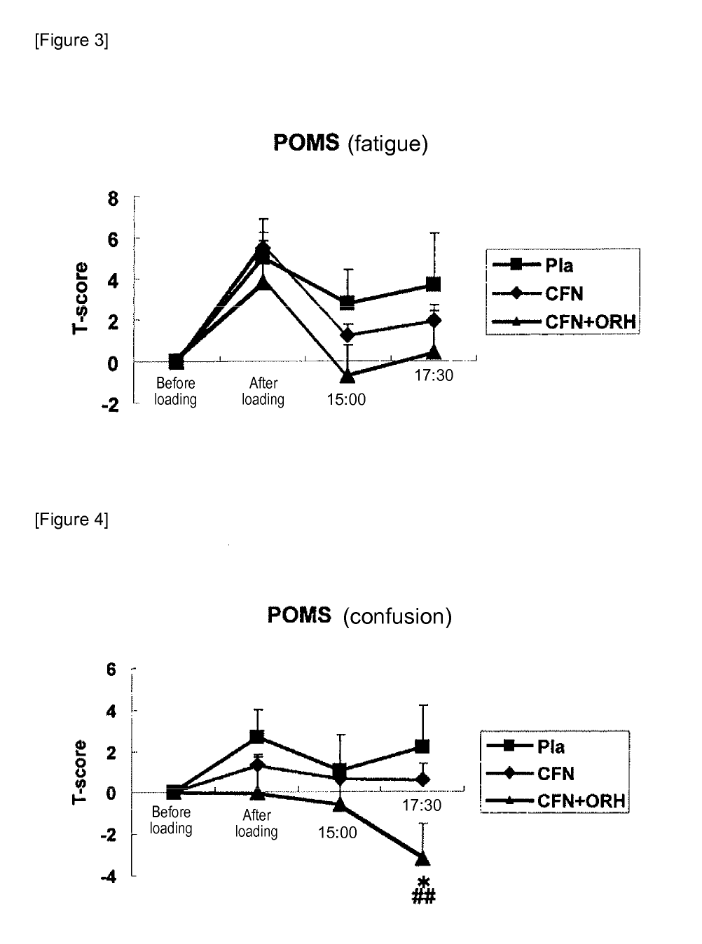

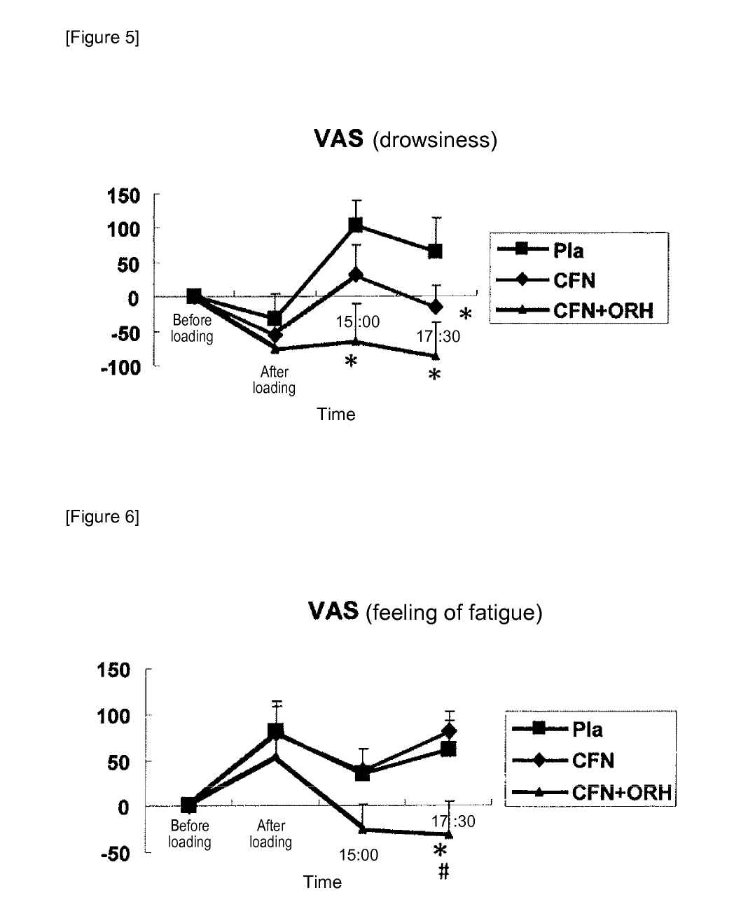

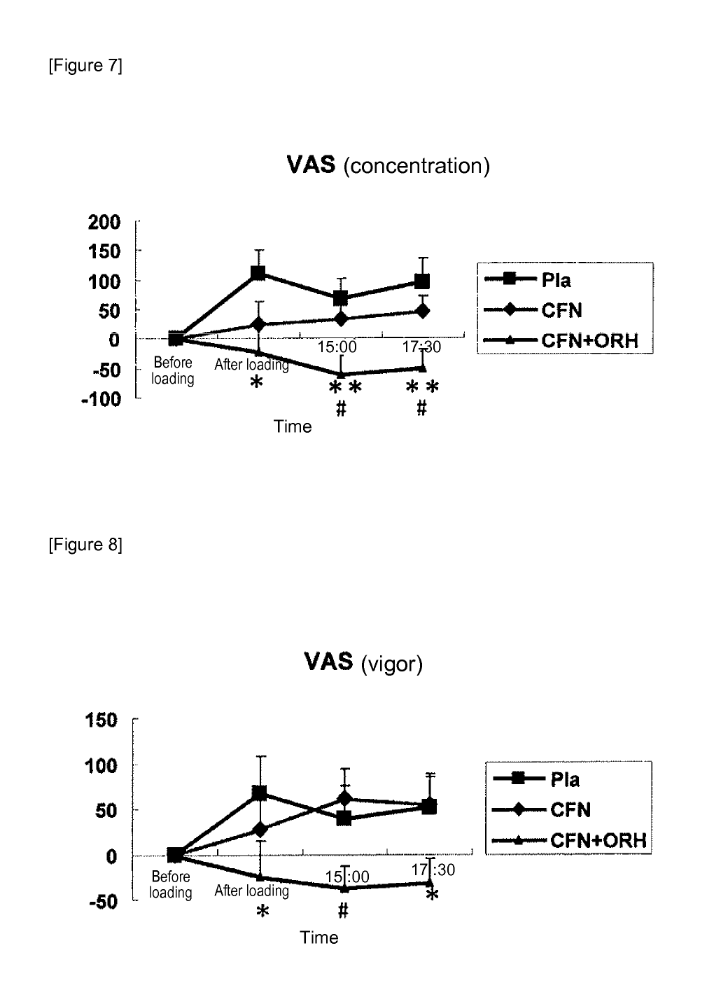

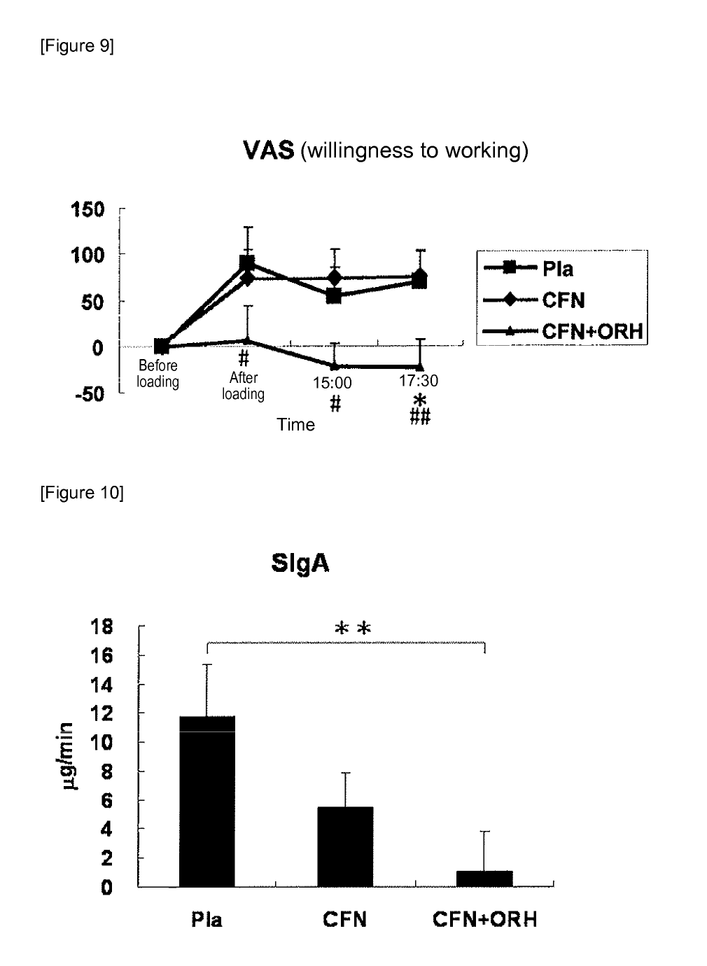

FIG. 2 shows a change in POMS “vigor” from the value before the calculation load, FIG. 3 shows a change in POMS “fatigue” from the value before the calculation load, and FIG. 4 shows a change in POMS “confusion” from the value before the calculation load. FIG. 5 shows a change in VAS “drowsiness” from the value before the calculation load, FIG. 6 shows a change in VAS “feeling of fatigue” from the value before the calculation load, FIG. 7 shows a change in VAS “concentration” from the value before the calculation load, FIG. 8 shows a change in VAS “vigor” from the value before the calculation load, and FIG. 9 shows a change in VAS “willingness to working” from the value before the calculation load. In addition, FIG. 10 shows a change in the amount of SIgA (secretory IgA) in saliva, which is considered to increase under a stressed condition, from the value before the calculation load.

As shown in FIGS. 2 to 4, in the group in which the subjects ingested both caffeine and ornithine hydrochloride, general sensations such as “vigor”, “fatigue” and “confusion” were improved from the time point after completion of the calculation load until 15:00 and 17:30 p.m., in comparison with other groups. These results show that fatigue after completion of the mental workload was alleviated and vitality was enhanced by a combined ingestion of caffeine and ornithine, in comparison with the case of ingesting caffeine alone.

Moreover, as shown in FIGS. 5 to 9, in the group in which the subjects ingested both caffeine and ornithine, general sensations such as “drowsiness”, “feeling of fatigue”, “concentration”, “vigor” and “willingness to working” were improved from the time point after completion of the calculation load until 15:00 p.m. and 17:30 p.m., in comparison with other groups. These results support the results of POMS, and also show that drowsiness and concentration were also improved by a combined ingestion of caffeine and ornithine, in comparison with the case of ingesting caffeine alone.

Furthermore, as shown in FIG. 10, an increase in the amount of SIgA was suppressed in the group in which the subjects ingested both caffeine and ornithine, more strongly than in other groups. These results are objective biochemical data demonstrating the results of the questionnaires shown in FIGS. 2 to 9.

### INDUSTRIAL APPLICABILITY

According to the present invention, when utilized various types of physiological action of caffeine which involve stimulation of the central nerve to promote mental function, such as mental fatigue-reducing action, drowsiness-preventing action or concentration-maintaining action, effective action effects can be obtained even from a low intake of caffeine, and further, the sustained action effects of caffeine can be obtained. Accordingly, the present invention provides: a method for safely and effectively enhancing physiological action of caffeine; and a composition for improving a physiological function comprising, as an active ingredient, the caffeine whose physiological action has been enhanced.

1. A method for enhancing physiological action of caffeine, comprising combining caffeine with ornithine or a salt thereof.

2. The method for enhancing physiological action of caffeine according to claim 1, wherein caffeine is combined with ornithine or a salt thereof in an amount equivalent to 0.1 to 100 parts by weight of free ornithine based on 1 part by weight of caffeine.

3. The method for enhancing physiological action of caffeine according to claim 1, wherein the physiological action of caffeine is mental fatigue-reducing action, drowsiness-preventing action, or concentration-maintaining action.

4. A composition for improving a physiological function comprising caffeine as an active ingredient, wherein ornithine or a salt thereof is added to caffeine as an agent for enhancing physiological action of caffeine in an amount corresponding to 0.1 to 100 parts by weight of free ornithine based on 1 part by weight of caffeine.

5. The composition for improving a physiological function comprising caffeine as an active ingredient according to claim 4, wherein the improvement of a physiological function by caffeine is the improvement of a physiological function by mental fatigue-reducing action, drowsiness-preventing action, or concentration-maintaining action.
